(19) United States
(12) Patent Application Publication
(10) Pub. No.: US 2025/0255657 A1
(43) Pub. Date: Aug. 14, 2025

(54) BALLOON CATHETER INFLATION SEQUENCING

(71) Applicant: Focus Medical Company, LLC, Edmond, OK (US)

(72) Inventor: Calvin Magee, Edmond, OK (US)

(73) Assignee: Focus Medical Company, LLC, Edmond, OK (US)

(21) Appl. No.: 19/097,953

(22) Filed: Apr. 2, 2025

Related U.S. Application Data
(63) Continuation-in-part of application No. 17/690,457, filed on Mar. 9, 2022, now Pat. No. 12,290,449.
Application No. 17/690,457 is a continuation-in-part of application No. 17/384,702, filed on Jul. 23, 2021, now Pat. No. 11,298,238.

(51) Int. Cl. A61B 17/88; A61M 25/10

(52) U.S. Cl. CPC A61B 17/8805; A61M 25/1011; A61M 25/10184; A61M 25/104; A61M 2025/1013; A61M 2025/1015; A61M 2205/6063; A61M 2210/1003

(57) ABSTRACT

A balloon catheter-type device and method of using the same. The device includes a balloon structure supported at a distal end of an extension tube. The balloon structure is configured for advancement through an incision in a patient to a target location within the patient, and has at least first and second inner balloons retained within an outer balloon. First and second fluid channels extend along the extension tube and are coupled to the respective first and second inner balloons. An inflation sequence can be used in which the first balloon is inflated followed by inflation of the second balloon. The outer balloon can remain uninflated or can be further inflated as well. Orientation indicia made of contrast response material can be used to identify a major or minor axis of the deployed balloon structure. A stylet wire can be used to deploy and retract the balloon structure.

### RELATED APPLICATIONS

The present application is a continuation-in-part of co-pending U.S. patent application Ser. No. 17/690,457 filed Mar. 9, 2022, which in turn is a continuation-in-part of U.S. patent application Ser. No. 17/384,702 filed Jul. 23, 2021 and now issued as U.S. Pat. No. 11,298,238. The contents of each of these applications are hereby incorporated by reference.

### FIELD OF THE DISCLOSURE

The present disclosure is generally directed to medical devices, and more particularly to balloon catheter devices and methods useful in a variety of areas including, but not limited to, kyphoplasty and angioplasty.

### BACKGROUND

In general, balloon kyphoplasty is a modified vertebroplasty technique. It is a minimally invasive procedure that aims to relieve pain, restore vertebral height, and correct kyphosis. During this procedure, an inflatable bone tamp (balloon catheter) is inserted into the collapsed vertebral body. Once inflated, the balloon elevates the end plates creating lift and thereby restores the height of the vertebral body. The balloon moves or shifts the pieces of broken or compressed vertebral bone and forms a space, i.e., a bony void when it is inflated. The balloon is deflated and removed, and the bony void is filled with bone cement.

The following presents a simplified summary of the innovation in order to provide a basic understanding of some aspects of the present disclosure. This summary is not an extensive overview of the present disclosure. It is intended to neither identify key or critical elements of the present disclosure nor delineate the scope of the present disclosure. Its sole purpose is to present some concepts of the present disclosure in a simplified form as a prelude to the more detailed description that is presented later.

### SUMMARY

Various embodiments of the present disclosure are generally directed to systems and methods for performing a balloon based medical procedure upon a patient using a specially configured balloon structure (catheter).

Without limitation, some embodiments provide a balloon catheter-type device having a balloon structure supported at a distal end of an extension tube. The balloon structure is configured for advancement through an incision in a patient to a target location within the patient, and has at least first and second inner balloons retained within an outer balloon. First and second fluid channels extend along the extension tube and are coupled to the respective first and second inner balloons.

An inflation sequence can be used in which the first balloon is inflated followed by inflation of the second balloon. The outer balloon can remain uninflated or can be further inflated as well. Orientation indicia made of contrast response material can be used to identify a major or minor axis of the deployed balloon structure. A stylet wire can be used to deploy and retract the balloon structure.

These and other features and advantages of various embodiments can be understood from a review of the following detailed description in conjunction with a review of the accompanying drawings.

### BRIEF DESCRIPTION OF DRAWINGS

FIG. 1 is an exemplary balloon kyphoplasty surgical device constructed and operated in accordance with various embodiments.

FIGS. 2A-2F depict various top cross-section cutaway views of exemplary balloon arrangements of the device of FIG. 1 in accordance with some embodiments.

FIGS. 2G-2I depict front perspective cutaway views of exemplary balloon arrangements of the device of FIG. 1 in accordance with further embodiments.

FIG. 3 is an enlarged cutaway side view of an exemplary port arrangement.

FIG. 4 is a side cross sectional view of an exemplary dual port/channel configuration of the port arrangement with one (1) port/channel for the four (4) inner balloons and one (1) port/channel for the outer balloon.

FIG. 5 is a side cross sectional view of an exemplary multi-port/channel configuration of the port arrangement having five (5) port/channels with one (1) port/channel for each of the four (4) inner balloons and one (1) port/channel for an outer balloon.

FIG. 6 is a top cross-sectional view and schematic of an exemplary single port/channel terminating in four (4) airway branches in fluid connection with the four (4) inner balloons.

FIG. 7 is a top cross-sectional view and schematic of an exemplary single port/channel terminating in fluid connection with the outer balloon.

FIG. 8 is a flow diagram of an exemplary kyphoplasty surgical process carried out in accordance with various embodiments of the present disclosure.

FIG. 9 depicts another exemplary balloon kyphoplasty surgical device in accordance with further embodiments.

FIGS. 10A-10B show respective isometric and exploded views of another exemplary balloon kyphoplasty surgical device in accordance with some embodiments.

FIGS. 11A-11B show respective cross-sectional and isometric exploded views of a stylet assembly of the device of FIG. 10 in accordance with further embodiments.

FIGS. 12A-12D show a balloon deflation sequence carried out using the stylet assembly of FIGS. 11A-11B in some embodiments.

FIG. 13 is a functional block diagram of an irradiation system that uses visually detectable orientation indicia on a target balloon kyphoplasty surgical device in accordance with further embodiments.

FIGS. 14A-14B provide schematic representations of exemplary orientation indicia that can be applied to the target device of FIG. 13 to identify respective major and minor axes thereof in accordance with further embodiments.

FIGS. 15A-15E show aspects of an exemplary balloon inflation sequence from an initial inflation state to a final inflation state in accordance with further embodiments.

FIG. 15F shows an alternative final inflation state for the sequence of FIGS. 15A-15E in accordance with further embodiments.

### DETAILED DESCRIPTION

It is to be understood that the specific devices and processes illustrated in the attached drawings and described in the following specification are exemplary embodiments of the inventive concepts defined in the appended claims. Hence, specific dimensions and other physical characteristics relating to the embodiments disclosed herein are not to be considered as limiting, unless the claims expressly state otherwise.

As shown in FIG. 1, an exemplary balloon kyphoplasty surgical device 10 includes an extrusion tube 15 having at least two internal fluid channels 21a, 22. Although only two internal channels 21a, 22 are illustrated in FIGS. 1, 3 and 4, it should be appreciated that additional channels may be contained within the extrusion tube 15 as illustrated in FIG. 5. For reference, the extrusion tube is also sometimes referred to as an extension tube.

In one embodiment, the extrusion tube 15 includes five (5) internal fluid channels described in more detail below. Housed within a length of the extrusion tube 15 is a support wire 25 (also sometimes referred to as a stylet). Positioned on a distal end 15a of the extrusion tube 15 is a balloon arrangement 30 in fluid communication with the at least two fluid channels 21a, 22. Positioned on a proximal end 15b of the extrusion tube 15 is a port arrangement 35 in fluid communication with the at least two (2) fluid channels 21a, 22 and a termination cap 36.

With the balloon kyphoplasty surgical device 10, when the balloon arrangement 30 is inflated, the balloon arrangement lifts with a predetermined shape resulting from the shapes of individual balloons comprising the balloon arrangement.

The balloon arrangement 30, also sometimes referred to as a balloon structure or a balloon assembly, may comprise a plurality of individual balloons. The plurality of individual balloons may comprise one or more inner balloons and at least one outer balloon. The outer balloon may be inflatable or remain uninflated. The outer balloon may be manufactured to have a predetermined shape. The outer balloon may be manufactured to retain the predetermined outer balloon shape when the outer balloon is inflated. The outer balloon may be substantially cylindrical, spherical, cubical, rectilinear, or any other suitable shape.

The one or more inner balloons are retained by the outer balloon and are configured to be inflatable or may remain uninflated. The inner balloon(s) may be manufactured to have a predetermined shape. Each inner balloon may have the same shape or the respective inner balloons can have different shapes. Each inner balloon may be manufactured to retain the predetermined inner balloon shape when the inner balloon is inflated. Each inner balloon may be substantially spherical, cylindrical, cube shaped, pyramid-shaped, oval, elliptical, rectilinear, wedge-shaped, or any other suitable shape.

The outer balloon may be inflated to a volume and/or pressure different from the volume and/or pressure of one or more inner balloons. One or more inner balloons may be inflated to a volume and/or pressure different from one or more of the other inner balloons. The balloon arrangement may have a cubic, cubic-type and/or rectilinear shape, rather than a circular-type shape, as is found in prior kyphoplasty devices. Other arrangements will readily occur to the skilled artisan in view of the present disclosure.

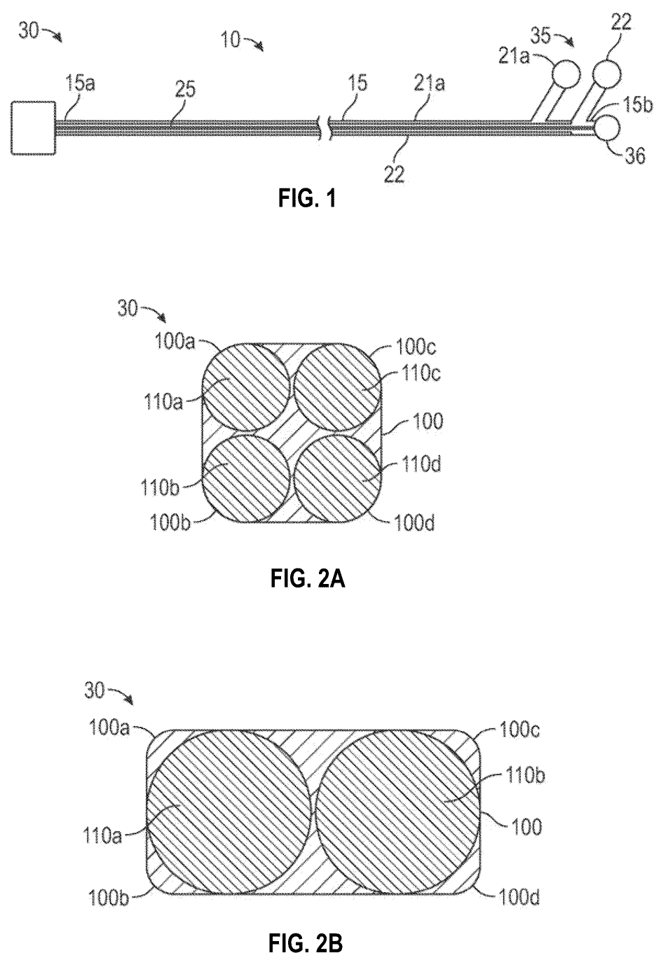

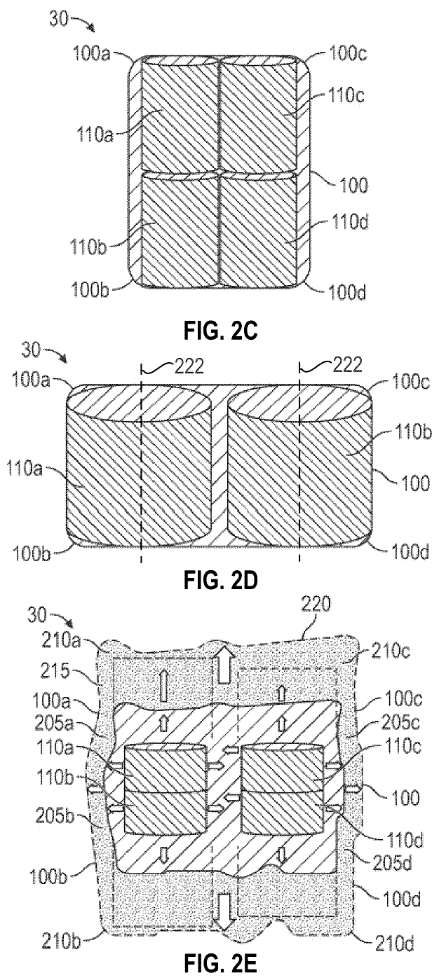

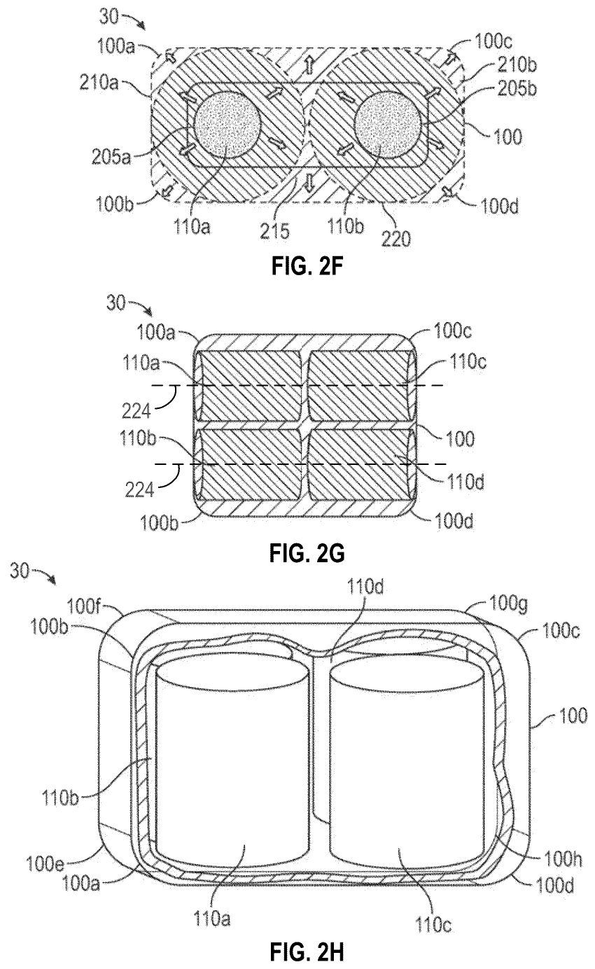

Referring now to FIG. 2A, a top cross-section cutaway view of the inflated balloon arrangement 30 is shown that enables the balloon arrangement to form a cubic-type shape. Here, the balloon arrangement 30 includes an outer balloon 100 that houses four (4) inner balloons 110a, 110b, 110c, and 110d. In the depicted balloon arrangement 30, the outer balloon 100 has been manufactured to be cubic in shape, while the inner balloons 110a, 110b, 110c, and 110d have been manufactured to each have a cylindrical and/or spherical shape and support the corners 100a, 100b, 100c, and 100d of the outer balloon 100 to maintain a cubic geometry of the inflated balloon arrangement 30 under pressure. Other configurations can be used.

It will be noted that the cubic-shaped outer balloon 100 has a total of eight (8) corners, so that additional corners 100e, 100f, 100g, and 100h are not shown in FIG. 2A but are represented in FIG. 2H. In another embodiment, various other geometric shapes of inner balloons and/or varying numbers of inner balloons may be combined to create the cubic shape of the kyphoplasty surgical device. For example, an exemplary kyphoplasty surgical device balloon arrangement implementation may be configured with two inner spherical balloons and one outer balloon as shown, for example, in FIG. 2B.

In FIG. 2B, the outer balloon 100 retains two inflated spherical inner balloons, 110a and 110b. Another exemplary kyphoplasty surgical device balloon arrangement implementation may be configured with four inner cylindrical balloons and one outer balloon as shown, for example, in FIG. 2C, illustrating the four inflated cylindrical inner balloons 110a, 110b, 110c, and 110d. Another exemplary kyphoplasty surgical device balloon arrangement implementation may be configured with two inflated inner cylindrical balloons 110a and 110b and one outer balloon 100 as shown, for example, in FIG. 2D.

Some balloon arrangement implementations in accordance with the present disclosure may include an inner balloon configured to be independently inflated to a pressure or volume different from other balloons in the balloon arrangement. In FIG. 2E, the four uninflated cylindrical inner balloons 110a, 110b, 110c, 110d are independently and separately inflated.

The cylindrical inner balloons 110a, 110b, 110c, 110d expand when inflated from respective uninflated volumes 205a, 205b, 205c, 205d to respective inflated volumes 210a, 210b, 210c, 210d, and the outer balloon 100 expands when inflated from an uninflated volume 215 to an inflated volume 220. In FIG. 2F, the two uninflated spherical inner balloons 110a and 110b are independently and separately inflated. The spherical inner balloons 110a and 110b expand when inflated from respective uninflated volumes 205a,205b to respective inflated volumes 210a, 210b, and the outer balloon 100 expands from the uninflated volume 215 to the inflated volume 220. In an illustrative example, a kyphoplasty surgical device balloon arrangement implementation in accordance with the present disclosure may comprise a variety of diverse balloon shapes and/or sizes.

A kyphoplasty surgical device balloon arrangement implementation in accordance with various embodiments of the present disclosure may comprise any number of inner balloons oriented as required to engage a patient's spine. As shown in FIG. 2D, one or more cylindrical inner balloons may be disposed with a balloon longitudinal axis 222 substantially parallel with a longitudinal axis of a patient's spine when the kyphoplasty surgical device is inserted into a collapsed vertebral body. For reference, a spine longitudinal axis 223 for a patient's spine 225 is shown in FIG. 2I.

Alternatively, as shown by FIG. 2G, one or more cylindrical inner balloons may be disposed with a balloon longitudinal axis 224 substantially perpendicular with the spine longitudinal axis 223 when the kyphoplasty surgical device is inserted into a collapsed vertebral body of the patient's spine 225. In the example depicted by FIG. 2G, the four inflated cylindrical inner balloons 110a, 110b, 110c, and 110d are further disposed with their respective longitudinal axes 224 perpendicular with a viewer's line of sight into the drawing sheet.

In some balloon arrangement implementations, a mix of balloon shapes may be configured to provide a customized contour improving the effective support of involved vertebrae. Inner balloons may be inflated to different pressures, to improve lift at different points on surfaces of involved vertebral bodies, to enhance the effectiveness of installation and improve the chance of a positive patient outcome. For example, a spherical balloon may be configured close to a cylindrical balloon, to provide support customized to an uneven surface of an involved vertebral body.

Although FIG. 2G depicts a front view illustrating a plurality of cylindrical inner balloons of a kyphoplasty surgical device balloon arrangement disposed with the inner balloon's longitudinal axes 224 substantially perpendicular with the longitudinal axis of a patient's spine, other orientations of the cylindrical balloons are possible. For example, an exemplary implementation may include one or more cylindrical inner balloon disposed in any orientation useful to improve treatment effect. For example, an exemplary cylindrical balloon may have two substantially parallel circular bases joined at a fixed distance by a curved surface. In an exemplary implementation, a cylindrical inner balloon may be oriented within an outer balloon to align at least one cylindrical inner balloon's base with an outer balloon's inner edge.

In an illustrative example using the kyphoplasty surgical device balloon arrangement 30 depicted by FIG. 2G in a front view, if the cylindrical inner balloons 110a, 110b, 110c, and 110d were rotated to align a base of each cylindrical inner balloon 110a, 110b, 110c, and 110d with the outer balloon's top inner edge, the cylindrical inner balloons 110a, 110b, 110c, and 110d respective top bases would appear in a top view as four circles, as depicted by FIG. 2A.

Aligning at least one cylindrical inner balloon's base with an outer balloon's inner edge may reduce the force per unit area supported by the cylindrical inner balloon, as a result of increased surface area engaged in load support. In an illustrative example, reducing the force per unit area supported by an inner balloon may help reduce the cost of kyphoplasty surgical device implementation by permitting the use of less expensive balloon material. In some implementations, reducing the force per unit area supported by an inner balloon may improve the effectiveness of stabilizing a collapsed vertebral body. Such improved vertebral body stabilization effectiveness may be a result of stabilization force from balloon inflation that is more evenly distributed over an increased area.

In an illustrative usage scenario of an exemplary kyphoplasty surgical device balloon arrangement 30 configured with four balloons, after insertion into the fractured area, the inner balloons 110a, 110b, 110c, and 110d and the outer balloon 100 are inflated. More specifically, the inner balloons 110a, 110b, 110c, and 110d are inflated to a size needed for the appropriate distance of lift, while the outer balloon 100 is inflated to have a volume of fluid added that is equal to the remaining volume needed to produce a cubic shape having 4 corners 100a, 100b, 100c, 100d for the inflated balloon arrangement 30. Inflation of the inner balloons 110a, 110b, 110c, and 110d and the outer balloon 100 is accomplished by the port arrangement 35.

In the exemplary balloon arrangement 30 depicted by FIG. 2H in a front perspective cutaway view, the eight (8) corners 100a-100g are visible, defining a cubic-type outer balloon 100 shape. The balloon arrangement 30 implementation depicted by FIG. 2H is shown for illustration purposes in a cutaway view to permit viewing the inflated inner balloons 110a, 110b, 110c, and 110d retained within the outer balloon 100.

In FIG. 2I, the exemplary balloon arrangement 30 is depicted with a portion of the human spine 225. In the illustrated example, the balloon arrangement 30 is positioned to lift or support an upper involved vertebra 230 and a lower involved vertebra 235. The depicted balloon arrangement 30 includes the inflated inner cylindrical balloons 110a, 110b, 110c, and 110d. In the depicted example, the inner balloon 110d is visible in perspective just behind the outer balloon 110c.

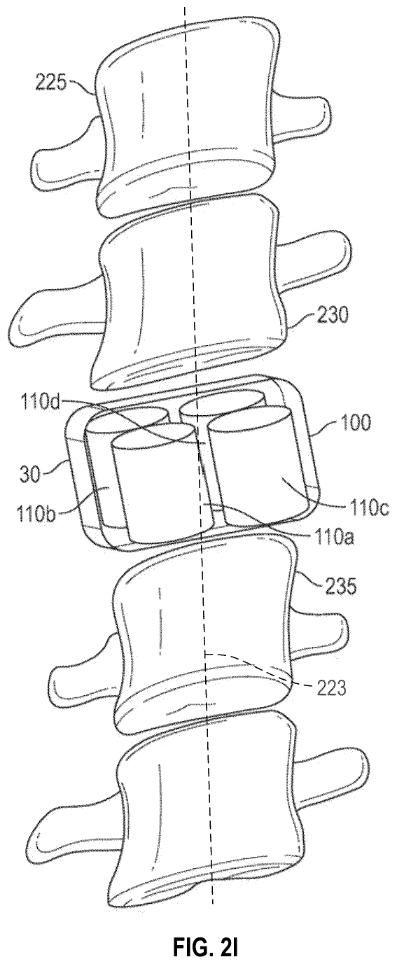

As shown in FIG. 3, the port arrangement 35 includes two ports 23a, 24 linked to respective chambers 21a, 22 (referred to interchangeably as channels or passages). In one embodiment, non-compressible fluid will be used to inflate the balloons such as but not limited to contrast fluid configured for X-ray or other irradiation source depiction. The two ports 23a, 24 are not only linked to but also in fluid communication with the respective chambers 21a, 22. Together, the ports and chambers are referred to as port/channels.

The ports 23a and 24 are also in fluid communication with inflation device reservoirs 40. The inflation device reservoirs 40 are controlled by a standard fluid pressure mechanism such as but not limited to valves working in concert with fluid pressure gauges and a balloon inflation device (which uses fluid) (not shown). In the depicted implementation, the inflation device reservoirs 40 retain contrast fluid.

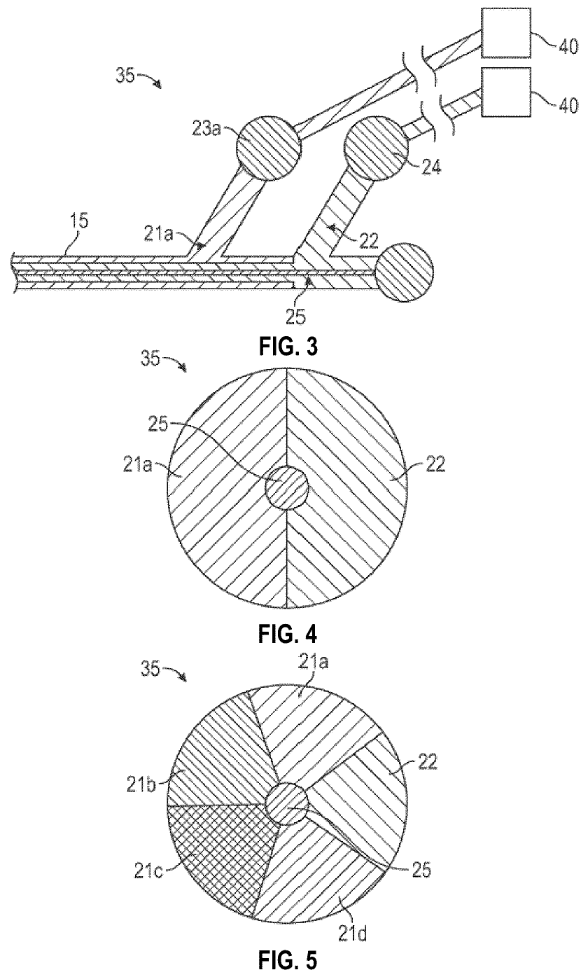

FIG. 4 illustrates a side cross sectional view of a dual port/channel configuration of the port arrangement with one port/channel 21a for the four inner balloons and one port/channel 22 for the outer balloon arranged about a central support wire 25.

As illustrated in FIG. 5, the port arrangement 35 includes one port/channel 21a, 21b, 21c, 21d for each of the four inner balloons and one port/channel 22 for an outer balloon. As before, the port/channels 21a-d and 22 surround a central support wire 25.

The presence of multiple ports/channels within the extrusion tube 15 enables an inflation device reservoir 40 controlled by standard fluid pressure gauges and valve mechanisms to maintain the pressure and volume used to inflate the respective inner and outer balloons that make up the balloon arrangement 30. In one embodiment, there are at least two (2) inflation device reservoirs 40 that independently maintain the pressure and volume used to inflate the respective inner and outer balloons that make up the balloon arrangement 30.

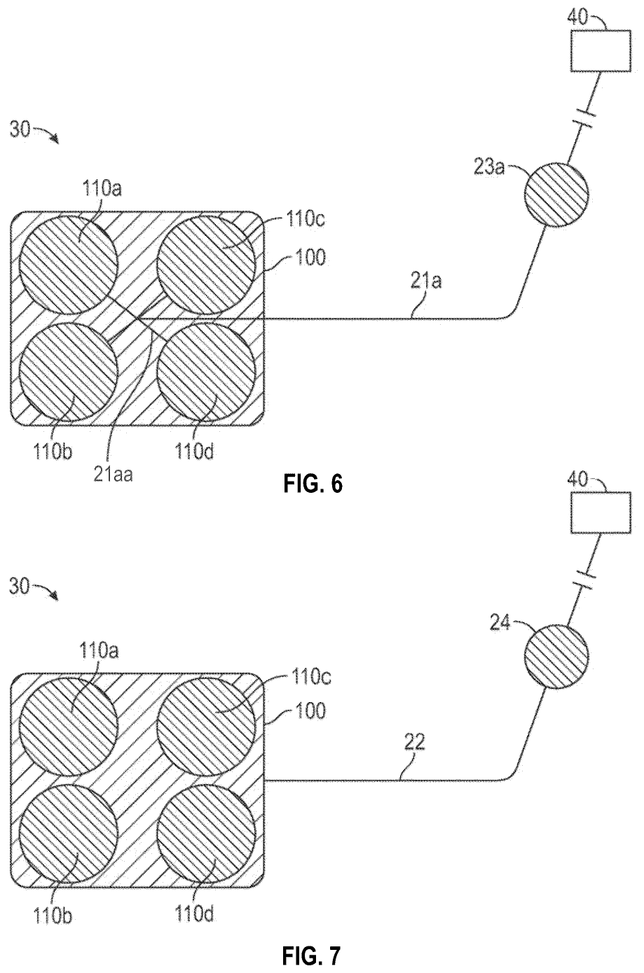

FIG. 6 is a top cross-sectional view and schematic of a single port/channel terminating in four airway branches 21aa in fluid connection with the four inner balloons. In the implementation depicted by FIG. 6, the four airway branches 21aa are four branches of the chamber 21a into respective inner balloons.

FIG. 7 is a top cross-sectional view and schematic of a single port/channel 22 terminating in fluid connection with the outer balloon.

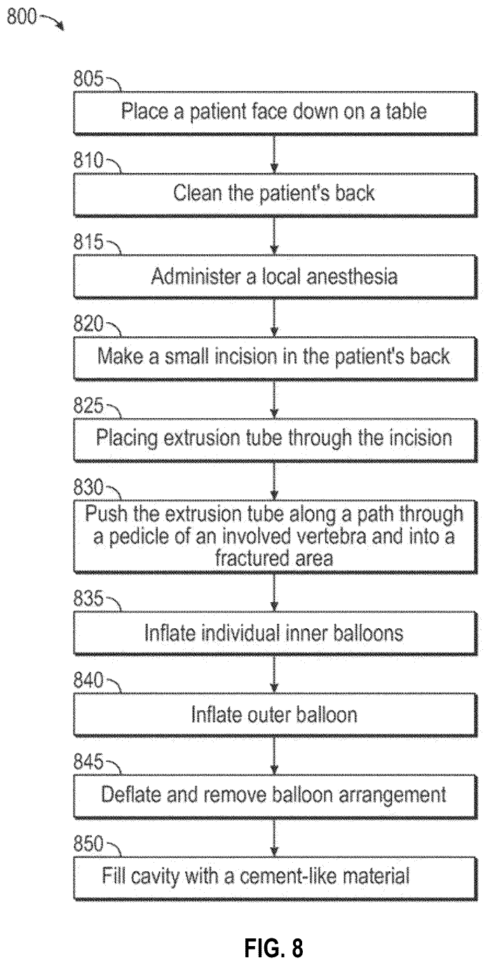

FIG. 8 is a flow diagram of an exemplary kyphoplasty surgical process 800. As shown in FIG. 8, the process commences with placing (805) a patient face down on a table. Further steps can include cleaning (810) the patient's back and administering (815) a local anesthesia; making (820) a small incision in the patient's back and placing (825) an exemplary kyphoplasty surgical device extrusion tube through the incision. The process 800 may include positioning an exemplary kyphoplasty surgical device to ensure a balloon is aligned to be square with the vertebra so the flat surface is parallel to the vertebra.

The kyphoplasty surgical process 800 further includes pushing (830) the extrusion tube along a path through a pedicle of an involved vertebra and into a fractured area. Once in the fractured area, inner balloons retained within an outer balloon of a balloon arrangement are inflated (835). As noted previously, the inner balloons may be spherically shaped, cylindrically shaped, or some other shape, and the outer balloon may be cubic shaped or some other shape.

Once the inner balloons are inflated, the outer balloon in the balloon arrangement is inflated (840), resulting in the balloon arrangement having a cubic shape, lifting the target area such as a vertebrae or fracture.

The balloon arrangement is then deflated and removed (845), and the cavity is gradually filled (850) with a cement-like filler material and allowed to harden.

The balloon arrangement of the present disclosure creates a larger cavity and a more structurally sound structure after cementation. During compression of vertebra, the bone is grown to exert this force through flat surfaces, and the cubic cement structure enables a flat surface for the bone to compress on and for more cement to be packed in. In one embodiment, the inflated balloon arrangement 30 is in place for 5 seconds to 30 minutes. The outer balloon may be sealed, unsealed, inflatable, or uninflatable, depending upon the requirements of a given application.

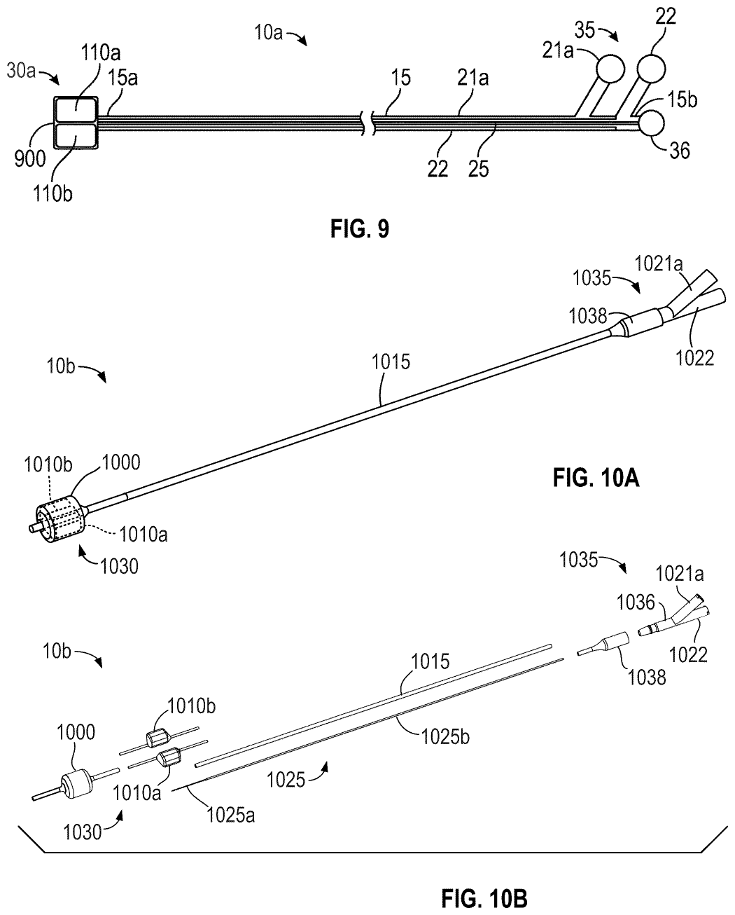

FIG. 9 shows another balloon kyphoplasty surgical device 10a with a sealed outer balloon configuration. As the device 10a is similar to the device 10 described above, like reference numerals are used for similar components.

The device 10a includes an extrusion tube 15 with at least two internal fluid channels 21a, 22 fluidically coupled to a balloon arrangement 30a. The balloon arrangement 30a includes two (2) inner balloons 110a, 110b and a sealed outer balloon 900. These respective balloons are shown in a deployed (partially or fully) inflated state. As before, any respective numbers of channels and balloons may be used as required, including the various embodiments discussed above.

A support wire 25 spans the length of the extrusion tube 15 from proximal end 15a to distal end 15b. The balloon arrangement 30a is fluidically coupled to the channels 21a, 22 at the proximal end 15a, and a port arrangement 35 is fluidically coupled to the channels 21a, 22 and a termination cap 36 at the distal end 15b.

In the illustrated implementation, the sealed outer balloon 900 constrains the expansion of the inflatable inner balloons 110a and 110b to create a more defined cubic-like shape when the inner balloons 110a and 110b are inflated. In an illustrative example the sealed outer balloon 900 may constrain the expansion of the inflatable inner balloons 110 a and 110 b with more force spread over a greater surface area than an inflatable outer balloon, improving the results of surgery and reducing a surgeon's effort stabilizing vertebral structures.

For example, using the sealed outer balloon 900 inner surface to constrain the expansion of the inflatable inner balloons 110a and 110b may increase the contact surface area between the inner balloons 110a and 110b with the sealed outer balloon 900 inner surface. Increasing the contact surface area between the inner balloons 110a and 110b and the sealed outer balloon 900 inner surface may reduce the force per unit area supported by an inner balloon and more evenly distribute force from the vertebrae over an increased area of the inner balloon, permitting more effective stabilization of the vertebral bodies.

Using the sealed outer balloon 900 inner surface to constrain the expansion of the inflatable inner balloons 110a and 110b may reduce a surgeon's effort adjusting the inflation of the inner balloons 110a and 110b to stabilize a collapsed vertebral body. When the inflatable inner balloons 110a and 110b inflate within the sealed outer balloon 900, the balloon arrangement 30 lifts with a cubic or cubic-type shape, in contrast with some prior kyphoplasty devices that may lift with a circular-type shape.

In FIG. 9, the sealed outer balloon 900 may house two or more inner balloons such as 110a, 110b. The sealed outer balloon 900 may be manufactured to conform to the shape of the two or more inner balloons. The inner balloons may be configured to have a cube shape to create the corners of the sealed outer balloon. Creating the corners of the sealed outer balloon using the cube shapes of the inflatable inner balloons helps to maintain a cubic geometry of the inflated balloon arrangement 30a under pressure.

Various other geometric shapes for the respective inner and/or outer balloons may be used to provide a desired final overall shape for the balloon arrangement 30a. After insertion into the fractured area, the inner balloons 110a, 110b may be separately or concurrently inflated to respective sizes needed for the appropriate distance of lift, while the sealed outer balloon 900 conforms around the inner balloons as they inflate, creating a square or other desired shape.

The sealed outer balloon 900 may be configured without a separate inflation port, so that the outer balloon is uninflatable and conforms to the inflation of the inner balloons. In alternative embodiments, the outer balloon 900 may be arranged to be partially or fully inflated during or after inflation of the inner balloons 110a, 110b.

FIGS. 10A and 10B show respective isometric and exploded views of another balloon kyphoplasty surgical device 10b similar to the devices 10, 10a described above. The device 10b includes a balloon arrangement 1030 with a substantially cylindrical outer balloon 1000 and offset square inner balloons 1010a, 1010b. An extension tube 1015 interconnects the balloon arrangement 1030 with a port arrangement 1035 formed from y-connection 1036 trocar 1038. As before, multiple channels/ports provide access to the balloons including ports 1021a, 10122.

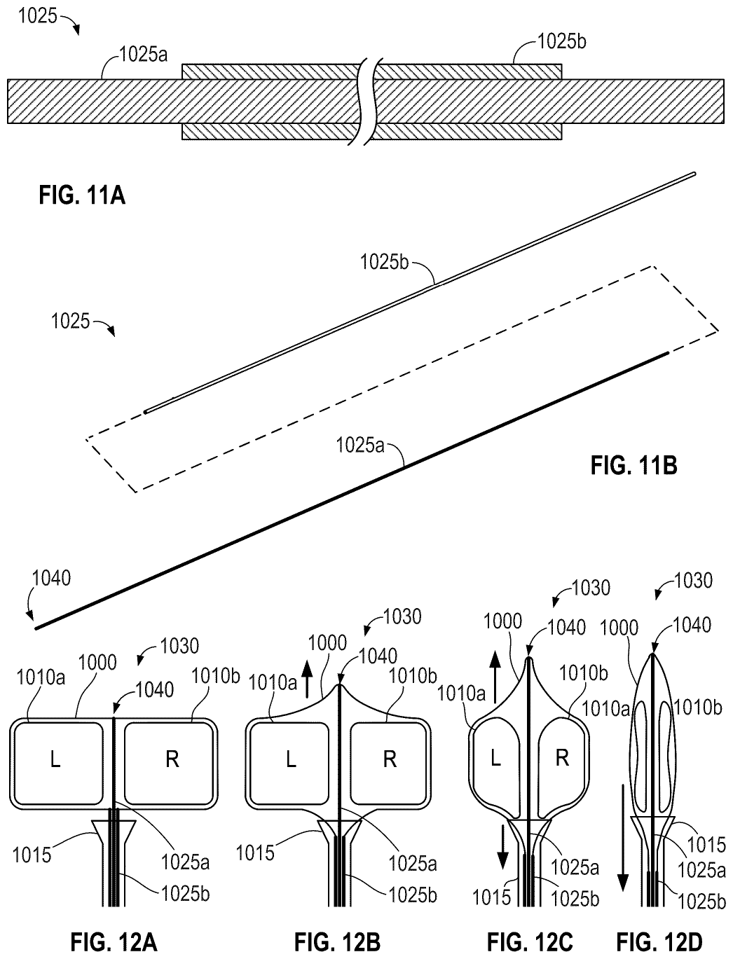

A stylet assembly 1025 includes an inner stylet wire 1025a which passes through an outer stylet tube (hypotube) 1025b, as further shown in FIGS. 11A and 11B. The inner stylet wire 1025a may be configured to be longer than the outlet stylet tube 1025b to allow retraction and extension of the inner wire with respect to the outer tube. In one nonlimiting example, the inner stylet wire 1025a may be on the order of about 12 inches, in. in length and the outer stylet tube 1025b may be on the order of about 10 in. in length. Other respective dimensions can be used.

The stylet assembly 1020 allows the user to apply tension to a deflated balloon structure, thus reducing the profile and allowing easier retraction of the balloon structure through the trocar. As will be appreciated, a balloon structure such as 1030 is initially packaged so as to be tightly wrapped or otherwise collapsed to allow the structure to pass from within the interior dimensions of the extension tube 1015 for deployment in the target area. Once the balloon structure has been inflated and subsequently deflated, it can be difficult in some cases to collapse the deflated balloon structure to a sufficiently small diameter to allow retraction back into the tube, particularly if some stretching of the balloon material has taken place during inflation.

Accordingly, FIGS. 12A through 12D show a balloon structure retraction sequence that can be carried out using the stylet assembly 1025 of FIGS. 11A-11B in some embodiments. It will be appreciated that FIGS. 12A-12D are simplified schematic drawings, so that various features will vary depending on the particular application.

In this example, the balloon arrangement 1030 from FIGS. 10A-10B includes left (L) and right (R) inner balloons 1010a, 1010b which are deployed in a side-by-side arrangement within outer balloon 1000. A distal end 1040 of the inner stylet wire 1025a is affixed to an inner surface of the outer balloon 1000 at an appropriate medial location of the balloon material. While not separately shown, a bracket, plate or other suitable attachment mechanism can be used to couple the outer balloon material to the inner stylet wire 1025a.

FIG. 12A shows the balloon structure 1030 in a deployed (inflated state). FIGS. 12B through 12D successively show deflation and retraction of the balloon structure 1030. As shown in FIG. 12B, during initial deflation of the inner balloons 1010a, 1010b and, if previously inflated, the outer balloon 1000, the user advances the inner stylet wire 1025a relative to the outer stylet tube 1025b to stretch the outer balloon material.

This forward movement of the stylet wire 1025a applies tension that allows the structure 1030 to attain a lower profile (e.g., smaller diameter), facilitating easier retraction into the distal end of the extension tube 1015 (see FIGS. 12C-12D). In some cases, all of the balloons 1000, 1010a, 101b may be connected to the stylet wire 1025a and be similarly elongated by the wire. In other cases, only some of the balloons, such as the outer balloon, may be so connected.

The tip 1040 of the stylet wire 1025a extends beyond the stylet hypotube 1025b that is fixed to a hub portion of the balloon structure. The stylet wire can terminate in a simple fitting that will allow the wire to be positioned in a retracted or extended position.

While it is contemplated that the inner wire 1025a and the outer hypotube 1025b may be closely spaced, an annular gap can be provided between these elements that is fluidically coupled to the internal cavity of the outer balloon. This gap can be used to inflate the outer balloon 1000 as required. Even if the outer balloon 1000 is uninflated, this gap allows a negative pressure (vacuum) to be applied to the interior of the outer balloon 1000, further aiding in collapse and retraction of the balloon structure 1030.

In some cases, the user (e.g., surgeon) may desire to position a given balloon structure in a particular axial orientation out of a number of available axial orientations. As discussed previously, a given balloon structure can have a larger length or other dimension along one axis as compared to another axis once the balloon structure is fully inflated in a deployed state. These different dimensions can advantageously provide the user with options with regard to how the balloon structure is inflated in order to obtain a desired overall separation distance for a given patient.

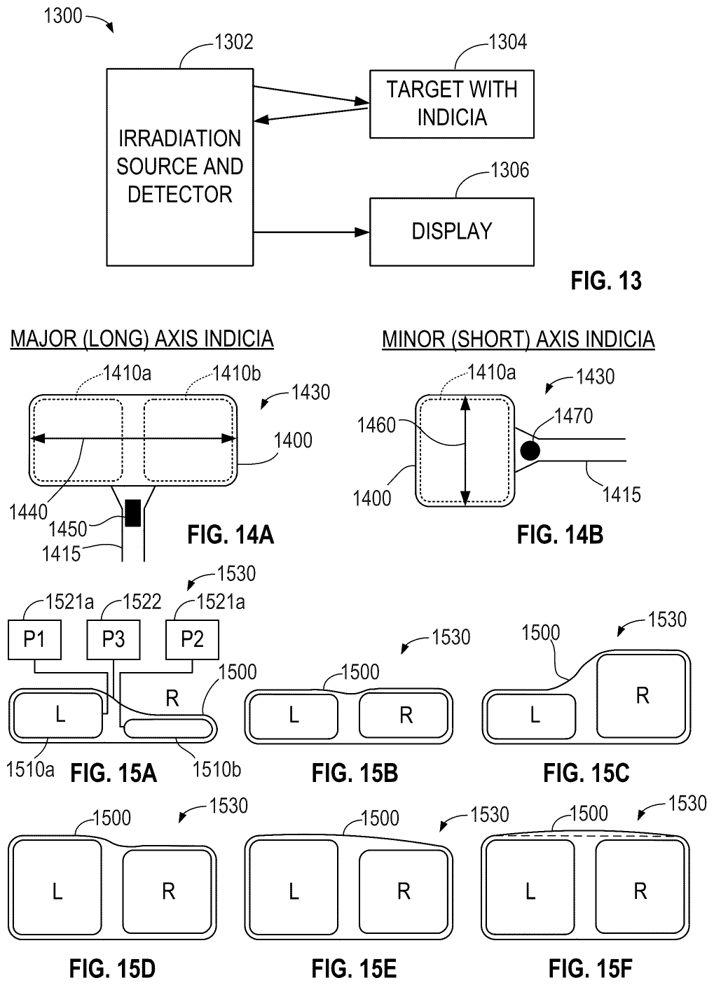

Accordingly, FIG. 13 shows a functional block representation of an irradiation system 1300 that can be used during deployment of a balloon kyphoplasty surgical device 10, 10a, 10b as variously embodied herein. An irradiation source and detector 1302 irradiates the interior of the patient to allow the user to observe manipulation of a target 1304 within the patient's body from one or more selected viewing angles. The target 1304 corresponds to the distal end of the surgical device, and includes visual indicia that can be detected and displayed visually on a display monitor 1306 as the target is manipulated by the user. In some embodiments, the irradiation source and detector 1302 may emit X-rays.

The visual indicia may be formed of indelible, detectable ink or other material that provides an absorptive or other contrast response on the monitor 1306 in a manner similar to the contrast fluid that may be used to inflate the respective inner and/or outer balloons. Any number of different types of distinguishing marks, symbols, arrows, lettering, lines or other indicia can be applied to the target 1304 to indicate a particular orientation for the balloon structure prior to and during deployment.

In some cases, the deployed balloon structure will have a substantially rectangular profile with distinct maximum (major) and minimum (minor) axes, as respectively shown in FIGS. 14A and 14B. More particularly, FIG. 14A shows a top-down view of an inflated balloon structure 1430 with outer balloon 1400, interior balloons 1410a, 1410b, and tube (hub) structure 1415. A longitudinally extending major (long) axis of the deployed balloon structure 1430 is indicated at 1440. It will be noted that this long axis 1440 may be parallel to, or perpendicular to, the longitudinal axes of the interior balloons 1410a, 1410b (see e.g., axis 222 in FIG. 2D, 224 in FIG. 2G).

A visual indicia mark 1450 is supplied to the distal end of the extension tube (hub) structure 1415. In this case, the mark is a rectangular mark. By viewing this mark from the top plan vantage point shown in FIG. 14A, the user can receive confirmation that the balloon structure will deploy in an orientation as shown in FIG. 14A. Other placements and orientations of the indicia can be provided as well, including on selected surfaces of the balloons or other elements, etc.

FIG. 14B shows a side plan view of the balloon structure 1430 from FIG. 14A. A minor (short) axis is denoted at 1460, and a short axis indicia mark 1470 is provided along this axis. In this case, the minor axis indicia 1470 is circular. By providing distinct indica along different axes, the user will be able to properly rotate the structure to the desired orientation prior to deployment. Moreover, by providing multiple indicia along different facing surfaces, correct orientation can be obtained regardless of the viewing angle supplied by the detection system 1300 (FIG. 13). Other suitable locations can be used as required.

Further embodiments of the present disclosure contemplate the controlled inflation of the respective balloons in a balloon structure using a controlled inflation sequence. As described previously, separate ports/channels can be provided to each of the inner balloons (and as desired, the outer balloon) to allow independent inflation of these respective balloons (see e.g., FIG. 5).

FIGS. 15A through 15E show an inflation sequence for another balloon structure 1530 in which the respective balloons are partially inflated in stages in order to controllably expand the target area in a controlled sequence. The balloon structure 1530 is similar to the structures discussed previously and includes an outer balloon 1500, inner balloons 1510a and 1510b, and respective ports 1521a, 1521b and 1522 (denoted as ports P1-P3).

For clarity, the inner balloons 1510a and 1510b are also referred to as left (L) and right (R) balloons in the following discussion. While only two inner balloons are used, it will be appreciated that other numbers of balloons can be incorporated into the balloon structure and subjected to similar processing.

The ports P1-P3 are operationally coupled to the respective left (L) and right (R) inner balloons, and to the outer balloon, via respective first, second and third fluid channels which are represented by solid line interconnecting paths. The fluid channels extend along an associated extension tube such as the tubes 15, 1015, 1415 described above. The tube supports the ports P1-P3 at a proximal end and the balloon structure 1530 at a distal end. The fluid channels extend in parallel within and along the length of the tube from the proximal end to the distal end thereof.

It will be appreciated that the inflation sequence may begin with all of the balloons in a fully deflated state as the assembly is maneuvered into position. FIG. 15A shows application of an initial partial inflation of the left (L) balloon 1510a to a first volume, while the right (R) balloon 1510b remains substantially uninflated. This is carried out by the application of pressurized fluid to the left (L) balloon via the first fluid channel. As described previously, the pressurized fluid may be a non-compressible X-ray responsive contrast liquid, or may be some other suitable fluid.

FIG. 15B shows the right (R) balloon has been partially filled with pressurized fluid using the second fluid channel during continued application of the inflation sequence. The pressurized fluid may be the same as, or different from, the pressurized fluid used in the left (L) balloon. In the nonlimiting example of FIG. 15B, the right (R) balloon has been filled to substantially the same fill volume/pressure as the left (L) balloon, so that both balloons provide a substantially uniform height distance. While not required, this stepwise filling can advantageously provide a controlled, even application of separation force to the associated vertebrae.

FIG. 15C shows continued application of the filling process with the right (R) balloon, bringing it to a next higher volume/pressure than that of the left (L) balloon. FIG. 15D shows additional filling of the left (L) balloon to a next higher level greater than that of the right (R) balloon. FIG. 15E shows application of pressurized fluid via the third fluid channel to the outer balloon 1500.

In the final state of FIG. 15E, it will be noted that the left (L) balloon has been inflated to a final volume/pressure greater than that of the right (R) balloon. This greater inflation of the left (L) balloon provides a greater separation distance and/or supporting force as compared to the right (R) balloon, and provides a contoured supporting surface shape. This is not limiting, however.

In an alternative embodiment, both the left (L) and right (R) balloons are inflated to substantially the same final volume/pressure, after which the outer balloon is inflated (or not, as required), as represented by an alternative final state in FIG. 15F. As such, each of the respective inner balloons can be ultimately inflated to the same or different levels (e.g., pressures, volumes, dimensions, etc.) as required by a given patient configuration, and the inflation sequencing can be applied as required to adjustably increase the separation distance established by the balloon assembly.

The separate, independent and sequential inflation of the inner balloons can be carried out in any respective order, including an alternating order as represented by FIGS. 15A-15F, to achieve a desired separation profile. This sequencing can include inflating each of the inner balloons to a succession of intermediate levels before achieving full inflation of each of the respective inner balloons at a final level. In this way, the sequencing and rate at which compressive force is applied by the balloon structure 1530 can be tuned, allowing the user to observe the expansion of the gap in real time and adaptively adjust the expansion as required.

While the various embodiments have used an even number of inner balloons within a single outer balloon, other arrangements are contemplated including an odd number of inner balloons (e.g., such as three balloons), multiple outer balloons enclosing sets of inner balloons, etc. In some cases, less than all of the available balloons may be inflated to achieve a final desired support configuration. These and other variations will readily occur to the skilled artisan in view of the present disclosure.

In some embodiments, the balloon structure as variously embodied herein can be inserted into the body of the patient using a 10f or 11f access needle or trocar that varies in length and size. The access needle or trocar is placed in the vertebral body and allows the balloon arrangement to enter through the needle or the trocar into the vertebrae. The access needle and trocar are not shown.

In sum, the balloon kyphoplasty surgical devices variously embodied herein may include multiple balloons, an extrusion tube, support wire, and a multichambered proximal port. The multiple balloons may be positioned as inner and outer balloons to produce a cubic shape when inflated. The one or more larger outer balloon may be manufactured to be cubic in shape, while the inner balloons may have a standard cylindrical/spherical shape and support the corners to maintain the square geometry under pressure. The extrusion tube may comprise multiple fluid channels so that the inner and outer balloons can be inflated at different volumes, different rates and different times. The support wire will ensure the device does not get damaged while inserting into the vertebra. The multichambered proximal port may comprise multiple ports that attach to separate chambers. These chambers connect to different fluid routes keeping the inlets separate, this separation will allow for independent filling during use.

It will be appreciated that a general goal of the balloon kyphoplasty surgical device as variously embodied herein is to achieve lift with a cubic like balloon shape or other suitable shape, including a generally rectangular shape with respectively deployable major and minor axes of different overall effective lengths. After insertion the inner and outer balloons are inflated, the inner balloons are inflated to the size needed for the appropriate distance of lift. The outer balloon may remain uninflated or may be inflated with a volume of fluid added to produce a final desired shape. The inflation may be carried out using multichambered ports and separate inflation device reservoirs to maintain the appropriate pressure/volume needed. While not limiting, the balloon material will be sufficiently robust in some applications to accommodate pressures of upwards of 400 PSI or more without losing shape or bursting while still being compliant enough to stretch to the appropriate size.

Kyphoplastic balloons of the existing art operate in a similar fashion but tend to use a standard circular balloon. The circular cavity created has proven to work but the cement structure formed after use can have high single point pressure areas that can weaken the bone at these points, these devices can also not allow for enough bone cement to be added for proper support. By contrast, the balloon kyphoplasty surgical device as variously embodied herein creates a larger boney void with a larger flat surface area producing a more structurally sound structure after cementation. During compression of vertebra the bone is grown to exert this force through flat surfaces, and the cubic cement structure allows for a flat surface for the bone to compress on and for more cement to be packed in.

While various embodiments have contemplated use of the balloon structure in the context of a spinal kyphoplasty environment, other applications for the balloon structure are contemplated including as a balloon angioplasty device. Angioplasty is a minimally invasive procedure performed to widen narrowed or obstructed blood vessels. The procedure accesses a blood vessel via a catheter inserted through an incision in the skin. Angioplasty is typically used to treat atherosclerosis caused by the buildup of plaque in a blood vessel but can also treat other conditions associated with narrowing or blockage of a blood vessel.

In an aspect, an exemplary implementation in accordance with the present disclosure may feature a balloon angioplasty surgical device similar to those described above and including an extrusion tube having internal fluid channels and a guidewire channel, a port arrangement positioned on a proximal end (Y connector/injection ports) of the extrusion tube and a balloon arrangement positioned on a distal end of the extrusion tube, the balloon arrangement resulting in a polygon shape when inflated by the port arrangement.

The inner balloons may be housed inside of the outer balloons and each balloon may be inflated separately at the least or some balloons may be paired up and inflated separately by group. The separate inflation is possible due to the proximal injection ports having separate chambers to allow for the balloons to inflate independently. The device is used for opening narrowed or obstructed blood vessels. The outer balloon may be pressurized first and the inner balloons may be pressurized separately based on the needs of the patient's artery. This allows for weaker areas of the vessels to not receive as much pressure.

In an aspect, an implementation in accordance with the present disclosure may feature a balloon angioplasty surgical device including an extrusion tube having internal fluid channels and guidewire channel, a balloon arrangement positioned on a distal end of the extrusion tube, the balloon arrangement resulting in a polygon shape when inflated by the port arrangement, and wherein there are three or more inner balloons.

The inner balloons may be housed inside of the outer balloons, each balloon may be inflated separately at the least, some balloons may be paired up and inflated separately by group or inflated all together. The separate inflation of the individual balloons is possible due to the proximal injection ports having separate chambers to allow for the individual balloons to inflate independently.

The proximal Y connection/Injection ports section may have at least two or more injection ports. One injection port may be configured for the guidewire channel, one injection port may be configured for the outer balloon, and other injection ports may be configured for the inner balloon. The device may be used for opening narrowed or obstructed blood vessels. The outer balloon may be pressurized first and the inner balloons may be pressurized separately based on the needs of the patient's artery. This allows for weaker areas of the vessels to not receive as much pressure. Prior technologies inflate a circular balloon across the entire area of the artery, some can inflate at different pressures axially but none of the existing technologies can inflate the arteries cross sectional area separately.

Various angioplasty balloon catheter implementations in accordance with teachings of the present disclosure may advantageously increase successful procedures and allow for limiting arterial damage as a result of an angioplasty balloon catheter designed to permit radial forces applied to the cross-sectional area of the artery to be customized based on the patient's needs. This facilitation may be a result of the use of inner balloons, individual fluid chambers that can inflate all the inner balloons separately or in groups or all together, a fluid chamber that can inflate the outer chamber, and a device configured to form an exemplary Polygon shape using inner and outer balloons (for example, a shape close to a circle formation but having some edges due to inner balloons).

Some exemplary angioplasty implementations in accordance with the teachings of the present disclosure may be configured with a various number of inner balloons and the inner balloons' internal configuration, size, and number may vary. Some examples may comprise more than three inner balloons. The number of ports may be greater than two, to allow for individual filling of inner balloons if needed. (For example, 6 balloons may be configured to be individually filled and include a guidewire port resulting in a design having 7 ports, or 6 balloons may be configured in groups of two filled separately and include a guidewire port resulting in a design having 4 ports). An implementation in accordance with the present disclosure may be configured with an injection port and Y connector designed to be separate or a combined entity. In some implementations the materials of the guidewire and extrusion tube may be interchanged. An exemplary angioplasty implementation in accordance with the present disclosure may be designed for inflation across the entire volume of the outer balloon and may be permanently installed in the outer balloon, and may also advantageously exert radial force differently across the cross sectional area of the artery, in contrast with some prior art devices that insert an inner balloon during the operation and disadvantageously inflate a smaller volume than the outer balloon while exerting equal force in all radial directions.

While various embodiments of the present disclosure have been described above, it should be understood that they have been presented by way of example only, and not of limitation. Likewise, the various diagrams may depict an example architectural or other configuration for the present disclosure, which is provided to aid in understanding the features and functionality that may be included in an exemplary implementation in accordance with the present disclosure. An exemplary implementation in accordance with the present disclosure is not restricted to the illustrated example architectures or configurations, but the desired features can be implemented using a variety of alternative architectures and configurations. Indeed, it will be apparent to one of skill in the art how alternative functional configurations can be implemented to implement the desired features of the present disclosure. Additionally, with regard to flow diagrams, operational descriptions and method claims, the order in which the steps are presented herein shall not mandate those various embodiments be implemented to perform the recited functionality in the same order unless the context dictates otherwise.

In the present disclosure, various features may be described as being optional, for example, through the use of the verb “may;” or, through the use of any of the phrases: “in some implementations,” “in some designs,” “in various implementations,” “in various designs,” “in an illustrative example,” or, “for example.” For the sake of brevity and legibility, the present disclosure does not explicitly recite each and every permutation that may be obtained by choosing from the set of optional features. However, the present disclosure is to be interpreted as explicitly disclosing all such permutations. For example, a system described as having three optional features may be implemented in seven different ways, namely with just one of the three possible features, with any two of the three possible features or with all three of the three possible features.

The phrases “connected to,” “coupled to” and “in communication with” refer to any form of interaction between two or more entities, including mechanical, electrical, magnetic, electromagnetic, fluid, and thermal interaction. Two or more components may be functionally coupled to each other even though they are not in direct contact with each other. The terms “abutting” or “in mechanical union” may refer to items that are in direct physical contact with each other, although the items may not necessarily be attached together. In various implementations, elements described herein as coupled or connected may have an effectual relationship realizable by a direct connection or indirectly with one or more other intervening elements.

Recitation in a claim of the term “first” with respect to a feature or element does not necessarily imply the existence of a second or additional such feature or element.

Although the disclosure is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described exemplary embodiments.

It is to be further understood that even though numerous characteristics and advantages of various embodiments of the present disclosure have been set forth in the foregoing description, together with details of the structure and function of various embodiments of the disclosure, this detailed description is illustrative only, and changes may be made in detail, especially in matters of structure and arrangements of parts within the principles of the present disclosure to the full extent indicated by the broad general meaning of the terms in which the appended claims are expressed.

What is claimed is:

1. A method comprising:
advancing, through an incision in a patient to a target location within the patient, a balloon structure comprising at least first and second inner balloons retained within an outer balloon, the balloon structure supported by a distal end of an extension tube;
inflating the first inner balloon with a pressurized fluid using a first fluid channel extending along the extension tube; and
subsequently inflating the second inner balloon with a pressurized fluid using a different, second fluid channel extending along the extension tube.

2. The method of claim 1, wherein the target location is adjacent at least one vertebra of the patient, the inflating of the first and second balloons exerts a force against the at least one vertebra during a kyphoplasty process, and the method further comprises subsequently injecting the target location with a cement-like filler material during the kyphoplasty process.

3. The method of claim 1, wherein the target location is adjacent at least one artery of the patient, and the inflating of the first and second balloons exerts a force against an arterial wall of the patient at the target location during an angioplasty process.

4. The method of claim 1, wherein the first and second balloons are alternately inflated to each of a succession of intermediate levels until each of the first and second balloons are fully inflated at a respective final level.

5. The method of claim 1, further comprising a subsequent step of inflating the outer balloon with a pressurized fluid using a different, third fluid channel extending along the extension tube after the first and second balloons are partially or fully inflated.

6. The method of claim 1, further comprising subsequent steps of:
deflating the first and second balloons; and
retracting the first and second balloons and the outer balloon into the distal end of the extension tube.

7. The method of claim 1, wherein each of the first and second inner balloons, when inflated, takes a substantially spherical, cylindrical, or offset square shape within the outer balloon.

8. The method of claim 1, further comprising using a stylet wire that extends along the extension tube to deploy the balloon structure at the target location, the stylet wire having a distal end affixed to the balloon structure and configured to move relative to the extension tube.

9. The method of claim 1, further comprising using an irradiation system to detect an orientation indicia on a selected surface of the distal end of the extension tube, the orientation indicia formed from indelible material that provides a contrast response on a display monitor of the irradiation system to identify a selected one of a a major axis or a minor axis of the balloon structure during deployment of the balloon structure at the target area.

10. The method of claim 1, wherein a proximal end of the extension tube supports a multi-port connector having a first port and a second port, wherein the balloon structure is inflated and deflated using the first port, and wherein the balloon structure is advanced and retracted relative to the distal end of the extension tube using a stylet wire that extends through the second port.

11. The method of claim 1, wherein the pressurized fluid used to respectively inflate the first and second inner balloons comprises a non-compressible X-ray responsive contrast liquid.

12. A device, comprising:
an extension tube having a proximal end and an opposing distal end;
a balloon structure supported by the distal end of the extension tube, the balloon structure comprising at least first and second inner balloons retained within an outer balloon;
a first fluid channel extending within the extension tube from the proximal end of the extension tube to the first balloon; and
a second fluid channel extending within the extension tube from the proximal end of the extension tube to the second balloon, the first and second fluid channels configured to facilitate separate inflation of the first and second inner balloons by steps comprising:
inflating the first inner balloon with a pressurized fluid using the first fluid channel; and
subsequently inflating the second inner balloon with a pressurized fluid using the second fluid channel.

13. The device of claim 12, further comprising a third fluid channel extending within the extension tube from the proximal end of the extension tube to the outer balloon and configured to facilitate inflation of the outer balloon with the pressurized fluid after inflation of the first and second inner balloons.

14. The device of claim 12, wherein the first and second balloons are alternately inflated to each of a succession of higher internal pressure levels until each of the first and second balloons are fully inflated at a respective final internal pressure level.

15. The device of claim 12, further comprising a port connector coupled to the proximal end of the extension tube and a stylet wire that extends through the extension tube, the stylet wire having a first end coupled to the balloon structure and a second end extending through a port of the port connector to facilitate deployment and retraction of the balloon structure.

16. The device of claim 12, wherein the outer balloon is characterized as an uninflatable sealed balloon.

17. The device of claim 12, further comprising orientation indicia placed on a selected surface of the distal end of the extension tube, the orientation indicia formed from indelible material that provides a contrast response on a display monitor of an irradiation system to identify a selected one of a major axis or a minor axis of the balloon structure during deployment thereof at the target area.

18. The device of claim 12, wherein each of the first and second inner balloons, when inflated, takes a substantially spherical, cylindrical, or offset square shape within the outer balloon.

19. The device of claim 12, characterized as a balloon kyphoplasty surgical device.

20. The device of claim 12, characterized as a balloon angioplasty surgical device.

21. The device of claim 12, wherein the balloon structure further comprises a third inner balloon within the outer balloon.

22. The device of claim 12, wherein the balloon structure comprises at least four inner balloons disposed within the outer balloon.
